(19) United States
(12) Patent Application Publication
(10) Pub. No.: US 2009/0143450 A1
(43) Pub. Date: Jun. 4, 2009

(54) METHOD FOR TREATING CENTRAL PAIN SYNDROM OR FOR INDUCING CENTRALLY GENERATED PAIN IN AN ANIMAL MODEL

(75) Inventor: Scott Thompson, Baltimore, MD (US)

Correspondence Address: BROWDY AND NEIMARK, P.L.L.C.;624 NINTH STREET, NW, SUITE 300, WASHINGTON, DC, 20001-5303, US

(73) Assignee: UNIVERSITY OF MARYLAND, BALTIMORE, Baltimore, MD (US)

(21) Appl. No.: 11/913,746

(22) PCT Filed: May 5, 2006
(86) PCT No.: PCT/US06/17745

§ 371 Date: Nov. 25, 2008

Related U.S. Application Data
(60) Provisional application No. 60/678,296, filed on May 6, 2005.

(51) Int. Cl. A61K 31/40; A61K 31/421; A61K 31/19; C12Q 1/02; A61P 25/04; A61D 99/00

(52) U.S. Cl. 514/376; 514/425; 514/557; 435/29; 128/898

(57) ABSTRACT

The present invention provides a method for treating central pain syndrome in a mammal by administering an effective amount of a thalamic anticonvulsant compound. Also provided are methods for inducing centrally generated pain responses in an animal model and for screening and identifying a compound that inhibits T-type calcium channels.

### CROSS-REFERENCE TO RELATED APPLICATION

This application claims the benefit of priority from U.S. provisional application 60/678,296, filed May 6, 2005, the entire content of which is herein incorporated by reference.

### BACKGROUND OF THE INVENTION

1. Field of the Invention

The present invention relates to treatment of central pain syndrome and an animal model for central pain syndrome. The present invention also relates to screening and identifying compounds that inhibit T-type calcium channels.

2. Description of the Related Art

Central Pain Syndrome (CPS) is defined as pain resulting from a lesion or pathology in the spinal cord, brainstem, or forebrain (Bonica, 1991; Yezierski, 2000). CPS is a significant neurological problem, with estimates of up to 200,000 afflicted individuals in the US alone. The majority of patients with CPS have suffered a spinal cord injury, and as many as 30% of spinal injury patients go on to develop CPS, including some with complete transection. CPS is also a significant complication for 20% of patients with multiple sclerosis (Bonica, 1991). Stroke survivors also develop CPS, particularly if the thalamus is affected, a condition known as Dejerine-Roussy syndrome. CPS patients are resistant to opioid analgesia and other pharmaceutical or surgical therapies. The severity of the pain and the lack of effective treatments is demonstrated by the high incidence of suicide among CPS sufferers (Gonzales, 1995). The National Institute of Neurological Disorders and Stroke, National Institutes of Health (healthlink/mcw.edu/article/921391401.html; Jun. 29, 2000) describes central pain syndrome as a neurological condition caused by damage specifically to the central nervous system (CNS)—brain, brainstem, or spinal cord. The pain is steady and is usually described as a burning, aching, or cutting sensation. Occasionally there may be brief, intolerable bursts of sharp pain.

Central pain is characterized by a mixture of pain sensations, the most prominent being constant burning. Mingled with the burning are sensations of cold, “pins and needles” tingling, and nerve proximity (like that of a dental probe on an exposed nerve). The steady burning sensation is increased significantly by any light touch. Patients are somewhat numb in the areas affected by this burning pain. The burning and loss of touch appreciation are usually most severe on the distant parts of the body, such as the feet or hands. Pain may be moderate to severe in intensity and is often exacerbated by movement and temperature changes, usually cold temperatures.

Central pain syndrome may develop months or even years after injury or damage to the CNS. The disorder occurs in patients who have, or have had, strokes, multiple sclerosis, limb amputations, or brain or spinal cord injuries.

Generally pain medications provide little or no relief for those affected by central pain syndrome. Patients should be sedated and the nervous system should be kept quiet and as free from stress as possible. Central pain syndrome is not a fatal disorder. But for the majority of patients, the syndrome causes debilitating, intractable pain.

The best way to manage pain is to treat its cause. For example, whenever possible, the cause of pain from cancer is treated by removing the tumor or decreasing its size. To do this, the doctor may recommend surgery, radiation therapy, or chemotherapy. When none of these procedures can be done, or when the cause of the pain is not known, pain-relief methods are used.

The pain experienced by CPS patients is typically described as severe and excruciating, with an aching or burning quality, and is usually perceived as originating deep to the skin (Bonica, 1991). The level of pain is generally steady and unrelenting in its intensity. There are several aspects of the way pain is perceived in these patients that suggest clues as to the underlying mechanism. First, it comes from areas in and around the primary sensory loss produced by the lesion. That is, if the lesion produces sensory deficits in left lower extremities, then the pain will be perceived as originating from the lower left extremities. This type of pain has been termed “below level” pain because it is perceived as originating in regions of the body represented at spinal levels more candal than the level of the lesion. Second, the pain is exacerbated by cold or mechanical movement; a significant problem for patients undergoing physical therapy as a result of their precipitating injury. Third, many patients suffer from allodynia and hyperalgesia (Greenspan et al., 2004). That is, the severe pain can be elicited by stimuli that are not normally painful or are delivered at an intensity that would not normally be perceived as painful, as well as by weakly noxious stimuli that would not normally be perceived as intensely painful. Finally, there is a marked delay of many weeks to months between the occurrence of the injury and the onset of the pain, with the majority of CPS patients not experiencing the onset of pain until more than one month after injury (Tasker et al., 1991).

The delayed occurrence of CPS was also discovered to be a significant consequence of spinothalamic tract lesions produced neurosurgically in humans in an effort to reduce neuropathic peripheral pain (White and Sweet, 1969). Indeed, lesions of the anterolateral spinal cord, where spinothalamic afferents to the ventrobasal thalamus ascend, result in allodynia and central pain in humans (Triggs and Beric, 1992).

The cause of CPS is unknown, but attention has been focused on the thalamus for nearly a century because of the prevalence of CPS after infarcts involving the thalamus (Dejerine and Roussy, 1906). In human CPS patients, the receptive fields of thalamic relay cells in the ventrobasal complex are absent in regions representing the areas of sensory loss and are greatly expanded in nearby regions (e.g., Lenz et al., 1987 and 1989). There is also a reorganization of sensory modalities such that a weak stimulus that would normally produce a thermal sensation now induces pain (Lenz et al., 1998). Electrophysiological recordings have repeatedly demonstrated that the behavior of thalamic cells and circuits is pathologically altered in CPS. EEG recordings reveal a slowing of the normal thalamic rhythms (Gucer et al., 1978). This phenomenon is thus not unlike the altered EEG seen in childhood absence epilepsy. More strikingly, whereas neurons in the ventrobasal complex display regular action potential discharge at about 10 Hz in non-CPS patients, neurons in CPS patients fire brief bursts of high frequency action potentials separated by periods of inhibition, as detected with extracellular single unit recordings (Lenz et al., 1987 and 1989; Jeanmonod et al., 1993). This abnormal burst discharge is particularly prominent in regions corresponding to the parts of the patient's body with pain and loss of sensation. Bursting discharges in the thalamus are normally associated only with certain sleep states, suggesting that it may represent a fundamental sign of dysfunction in these patients. As expected if the thalamus is the site of origin of CPS, weak microstimulation in regions of the thalamus is capable of eliciting painful sensations in CPS patients, particularly when the stimulation is delivered in regions where excessive bursting is observed, but not in non-CPS patients (Davis et al., 1996; Lenz et al., 1987; 1998; Dostrovsky, 2000). Indeed, microstimulation in these regions is more likely to produce a painful sensation in CPS patients (Davis et al., 1996). In agreement with these electrophysiological changes, SPECT imaging revealed an increased blood flow to the VB thalamic nuclei during periods of pain perception in a CPS patient, consistent with a putative increase in metabolic demand due to increased bursting activity (Ness et al., 1998).

Nociceptors in the periphery convey information about noxious stimuli via primary afferent fibers terminating in the dorsal horn of the spinal cord. Pain related information is then transmitted in several ascending pathways (Willis and Coggeshall, 1991). Most important for the conscious perception of pain, however, is the spinothalamic tract (e.g., Peschanski et al., 1986). Physiological studies have shown that most spinothalamic tract neurons in the dorsal horn respond to both innocuous cutaneous stimuli and noxious stimuli (e.g., Besson and Chaouch, 1987; Owens et al. 1992). The axons of these spinothalamic tract cells decussate in the spinal cord, ascend in both anterior and lateral bundles of the spinal cord, and ultimately terminate in various nuclei of the thalamus, most importantly in the rat the ventrobasal complex (VB) (e.g., Besson and Chaouch, 1987; Giesler et al., 1981; Zemlan et al., 1978), including both ventroposterolateral and ventroposteromedial regions, as well as the posterior nuclei and the central lateral nuclei. It is also becoming clear that the dorsal column nuclei convey nociceptive information to the VB via a dorsal pathway and the medulla (e.g., Peschanski et al., 1983; Al-Chaer et al., 1996; Kanjander and Giesler, 1987; Ma et al., 1986). There are other sources of excitatory input to the VB, including trigeminal and reticular pathways, as well as corticofugal afferents.

Electrophysiological studies demonstrate that VB thalamocortical relay cells in rats respond robustly to both innocuous and noxious somatosensory and visceral stimuli (e.g., Waldron et al., 1989; Peschanski et al., 1980; Guilbaud et al., 1980).

As the author of an overview of CPS concluded (Casey, 1991), “It would be most useful if there were animal models of CPS that would allow not only a study of the pathology, but a testing of . . . various therapeutic measures.” There are scattered reports of alterations in pain sensation and thalamic excitability in primates and cats after partial or complete spinal transection. In general, these studies have reproduced the phenomena seen in the thalamus of CPS patients: immediate loss of somatosensation followed by the delayed onset of excessive thalamic bursting, return of nociception, and reorganization of somatosensory receptive fields (Vierck et al., 1990; Koyama et al., 1993; Weng et al., 2003). Abnormal responses were seen in areas with intact rostral receptive fields as well as areas representing partially deafferented ‘below level’ body surfaces (Weng et al., 2003). Microelectrode and electroencephalographic recordings have revealed increases in overall discharge levels in the thalamus, abnormal responsiveness to peripheral stimulation, and increases in the frequency of bursting discharges. Ethical and economic considerations have prevented a mechanistic examination of the thalamus in these animals, however. There are few descriptions of behavioral CPS after spinal cord injury in rat, but they do offer encouragement. Mechanical contusion (Hulsebosch et al., 2000) or local ischemia (Hao and Xu, 1996) of the spinal cord has been shown to produce delayed increases in pain sensitivity. Similarly, spinal hemisection was found to result in allodynia for tactile and thermal stimuli at 24 days post-injury (Christensen et al., 1996; Bennett et al., 2000a,b). It is difficult to determine with these models, however, whether the pain symptoms result from interruption of ascending spinothalamic afferent pathways, or rather from the severing of descending pathways that activate inhibitory intraspinal circuitry. Furthermore, no detailed electrophysiological analyses have been performed to test whether these injuries produce alterations in thalamic electrophysiology comparable to those seen in human CPS.

Potentiation of pain pathways in the spinal cord and brain stem is thought to contribute to several forms of neuropathic pain (Dubner and Ren, 2004). In the phenomenon known as sensitization, for example, an NMDA receptor (NMDAR)-dependent form of synaptic potentiation seems to result in a lasting facilitation of the excitation of dorsal horn neurons by incoming peripheral afferents (Willlis, 2002; Ji et al., 2003). Koyama et al. (1993) reported that the acute application of an NMDAR antagonist eliminated the abnormal electrophysiological hyperexcitability seen in VB relay cells after a transection of spinothalamic tract in cats. Similarly, Bennett et al. (2000b) found that intrathecal administration of NMDAR antagonists eliminates behavioral allodynia after spinal cord injuries in rats.

The electrophysiological bases of ‘bursting’ and ‘regular spiking’ types of action potential discharge in thalamic relay cells and thalamic synaptic networks have been well established through the study of ex vivo thalamic brain slices (McCormick, 1989; McCormick and Bal, 1994). In brief, bursting is mediated by low threshold Ca²⁺ spikes driven by so-called T-type voltage-dependent Ca²⁺ currents (Huguenard, 1996). The unique voltage-dependent activation and inactivation properties of these currents account for much of the electrophysiological behavior of the cells (Coulter et al., 1989). T-type currents are mediated by Ca²⁺ channels containing α1G subunits (Kim et al., 2003). These channels are activated with small depolarizations from the resting membrane potential and thus have a low threshold for activation. Once activated, however, they inactivate fairly rapidly (over ca. 50 ms), thus giving rise to a transient Ca²⁺ current. Under normal conditions, this current produces a transient depolarization of the cell membrane potential (ca. 10 mV, 50 ms) which triggers a burst of conventional, fast Na⁺- and K⁺-dependent action potentials at high frequencies. These responses are known as low threshold spike bursts. Once the channels are inactivated, a strong hyperpolarization is needed to remove the inactivation and return the channels to a state from which they can re-open. Because the activation and inactivation voltage thresholds are close to the resting membrane potentials of neurons, small changes in the resting membrane potential can have large effects on the ability of a cell to display T-type currents. When the membrane potential of a thalamic relay cell is more negative than about −65 mV, then the T-type channels are de-inactivated and available for opening. Under these conditions, small excitatory postsynaptic potentials (EPSPs) are sufficient to cause regenerative activation of T-type channels and trigger a low threshold spike burst. If the neuron is more depolarized than −65 mV, in contrast, the T-type Ca²⁺ channels are inactivated, and EPSPs generate only one or two fast spikes. Neuromodulatory transmitters, such as acetylcholine and norepinephrine, from afferent pathways originating in subthalamic structures produce relatively tonic changes in the membrane potentials of thalamic relay cells and thus determine whether or not T-channels are available for activation (Steriade, 2004). During waking states, relay cells are relatively depolarized so that incoming sensory information is faithfully relayed to the cortex as precisely timed fast action potentials. During some sleep states, in contrast, relay cells are hyperpolarized and they display low threshold spike bursts, giving rise to the rhythmic oscillatory activity prominent in EEG recordings.

Opposing the inward T-type Ca²⁺ current are the outward K⁺ currents. Two prominent currents have been described: transient (IA) and sustained (e.g. Huguenard and McCormick, 1992). A-type K⁺ currents are activated with small depolarizations from the resting membrane potential and then inactivate over 20-50 ms. Both activation and inactivation occur at more depolarized voltages than for T-type currents, although it is clear that they can affect the ability of thalamic cells to display low threshold spike bursts (Pape et al., 1994). Interestingly, A-type currents in some cells are known to be modified by changes in activity (Bernard et al., 2004; Frick et al., 2004). Sustained currents, by comparison, also activate with small depolarizations, but do not inactivate. Changes in the magnitude of K⁺ currents can affect the ability of a relay cell to fire low threshold spike bursts. When K⁺ currents are reduced, the induction of low threshold spike bursts is facilitated (Gutierrez et al., 2001).

The final current that plays a prominent role in determining the discharge of thalamic relay cells is the hyperpolarization-activated or H-type current. H-channels are permeable to Na⁺ and K⁺ in a manner that gives rise to an inward or depolarizing current at the resting membrane potential. They are activated at membrane potentials that are more negative than the resting membrane potential, and inactivate slowly at the resting potential and more depolarized voltages. The channels are thus normally closed at rest. They are activated by a hyperpolarization of the cell such as that produced by IPSPs, and they then cause a transient ‘rebound’, depolarization of the cell following the hyperpolarization. They thus act in a similar manner to T-type currents and contribute significantly to rhythmic thalamic oscillations (Lüthi and McCormick, 1999; Bal and McCormick 1996). H-current can also modified in some cells by changes in activity or by brain injury (Lüthi and McCormick, 1999; Shah et al., 2004).

Synaptic excitation of thalamic relay cells by sensory and corticothalamic afferents is mediated by glutamate, which can act on both NMDARs and AMPARs in the usual manner (e.g. Salt, 1986; Golshani et al., 1998). There appear to be differences in the probability that action potentials in various pathways successfully release glutamate, however, as can be inferred from differences in responses to paired stimuli (Alexander and Godwin, 2005).

GABAergic inhibition in thalamic relay cells is mediated by interneurons whose cell bodies are located in the nucleus reticularis thalami (nRT). These cells are excited by collateral branches of both descending corticofugal axons and ascending thalamocortical axons. Activation of nRT cells results in hyperpolarizing GABAergic IPSPs in relay cells. This hyperpolarization de-inactivates the T-currents of the relay cells and promotes bursting (von Krosigk et al., 1993). An increase in inhibition of relay cells thus, paradoxically compared to cortical tissues, promotes bursting in relay cells and results in a positive feedback increase in the excitation of nRT cells (Kim et al., 1997). Excessive bursting in thalamic relay cells increases the activation of nRT cells, thereby increasing feedback inhibition of relay cells and promoting further bursting. It has been postulated that this process underlies the generation of absence epilepsy (McCormick and Contreras, 2001; Timofeev and Steriade, 2004).

Slow rhythmic bursting of thalamic relay cells is the hallmark of childhood absence epilepsy (Panayiotopoulos, 1997). Ethosuximide is an effective, well tolerated, anticonvulsant for this form of epilepsy and is currently the standard first-line treatment, as well as valproic acid. Several drugs that effectively manage generalized absence epilepsy, including dimethadione, ethosuximide, methylphenylsuccinimide, and valproic acid, act, at least in part, by reducing low threshold Ca²⁺ currents (Coulter et al., 1990; Kelly et al., 1990). This action is not shared by other anticonvulsants that are ineffective in clinically reducing absence seizures. Presumably, the reduction in low threshold Ca²⁺ current reduces the amplitude and duration of low threshold spike bursts, and in this manner weakens the positive feedback loops between the relay nuclei and the nRT, thereby reducing absence seizures (Huguenard, 2002).

There is compelling evidence in both in human CPS and primate models of CPS that thalamic relay cells display excessive burst discharges. Whether low threshold Ca²⁺ currents, K⁺ currents, glutamatergic excitation, or GABAergic inhibition are altered in the thalamus after partial deafferentation, and account for the excess bursting, is not known. Ethosuximide and drugs effective against absence epilepsy have not been tested in CPS patients.

Traumatic brain injury and stroke are significant risk factors for epilepsy, as well as CPS. A striking characteristic of this posttraumatic epilepsy is the variable delay between the trauma itself and the development of seizures, which can last from weeks to years, much like CPS. The laboratory of the present inventor has used organotypic hippocampal slice cultures to develop an in vitro model of posttraumatic epilepsy that allows the mechanisms underlying the development of hyperexcitability to be investigated under carefully controlled in vitro conditions (McKinney et al., 1997). In this model, the Schaffer collateral axons of CA3 pyramidal cells in hippocampal slice cultures are transected after the cultures have been allowed to develop in vitro for >14 days. The area CA3 has been shown to has been shown to become hyperexcitable because of the sprouting of axons injured by the transection (McKinney et al., 1997). The laboratory of the present inventor has further shown that sprouting is mediated by injury induced neurotrophin secretion.

Because current therapies used to treat CPS are inadequate and offer little relief to the patient for extended periods of time, and further no cure for CPS exist, there remains a long-felt need in the art for improved methods useful in the treatment of CPS.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention provides a method for treating central pain syndrome (CPS) in a mammal by administering an effective amount of a thalamic anticonvulsant compound. This method fulfills an unmet need for an effective therapy for CPS.

The present invention also provides a method for inducing centrally generated pain responses in a non-human mammal as an animal model for CPS in humans.

Further provided by the present invention is a method of screening and identifying a compound that inhibits T-type calcium channels.

### BRIEF DESCRIPTION OF THE DRAWINGS

FIG. 1 shows responses of denervated CA1 cells and control cells to synaptic stimulation or dendritic glutamate photolysis.

FIG. 2 shows extracellular recordings of neuronal activity in thalamic brain slices from an animal whose spinothalamic tract was lesioned 19 days earlier (lower row), but not in slices from a sham control animal (upper row).

FIG. 3 is a graph showing the time course of allodynia in animals after spinothalamic tract transection, as assayed with the von Frey's test.

FIGS. 4A and 4B are graphs showing hyperalgesia (FIG. 4A) and allodynia (FIG. 4B) in animals with lesioned spinothalamic tracts.

### DETAILED DESCRIPTION OF THE INVENTION

Because current therapies for treating CPS are inadequate and offer little relief to a subject suffering from CPS for any extended period of time, and because no cure for CPS currently exists, there remains a long felt need in the art for methods useful in the treatment of CPS. The present invention fulfills this need by providing a method for treating CPS that involves administering to a subject in need thereof an effective amount of an anticonvulsant compound which reduces hyperexcitability of thalamic relay cells in the ventrobasal complex of the thalamus. The results from Example 1, presented hereinbelow, demonstrate that increased postsynaptic excitation, resulting from changes in the function of intrinsic voltage-dependent ion channels, contributes to lesion- and denervation-induced hyperexcitability.

Thalamic relay cells in the ventrobasal complex of the thalamus respond homeostatically to the decrease in afferent activation with a delayed increase in their excitability, which appears electrophysiologically as a prolongation of burst responses. The hyperexcitability results from increased intrinsic neuronal excitability (i.e., downregulation of K⁺ channels or upregulation of Ca²⁺ channels) and/or increased network excitability (altered inhibition). The excessive thalamic discharge in the partially denervated cells mimics the discharge generated by the thalamus during strong noxious stimuli and is therefore perceived in the cortex as intense pain.

Burst discharges are generated in the normal healthy thalamus by low threshold Ca²⁺ spikes mediated by T-type voltage-dependent Ca²⁺ currents. Membrane depolarization first activates these channels and then inactivates them within a few tens of milliseconds, generating a depolarizing envelope upon which a burst of fast, sodium-dependent action potentials are superimposed.

Anticonvulsant compounds, such as ethosuximide used to effectively treat childhood absence epilepsy, can be used to reduce the hyperexcitability (excessive bursting) of thalamic relay cells and treat CPS by inhibiting T-type calcium channels and reducing low threshold Ca²⁺ current. As will be appreciated by those of skill in the art, the CPS to be treated by the embodiment of the present invention can be the consequence from any number of causes, including but not limited to spinal cord injury, stroke, multiple sclerosis, etc.

Compounds that inhibit T-type calcium channels (but without affecting a cell's ability to generate a fast, sodium channel-mediated action potential) and thereby treat CPS are considered anticonvulsant compounds that act on the thalamus (thalamic anticonvulsants). Classes of compounds that are particularly effective as thalamic anticonvulsant compounds include succinimides and oxazolidinediones. Accordingly, preferred compounds suitable for use in the method embodiment for treating CPS include compounds of the formula

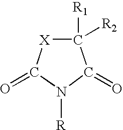

wherein: R is H or lower alkyl; R₁ and R₂ are independently selected from the group consisting of H, lower alkyl, aryl, and aryl lower alkyl; and X is —O— or —CH₂—, with the proviso that at least one of R, R₁, and R₂ is an indicated substitutent other than hydrogen. Lower alkyl preferably means a linear or branched alkyl group containing from 1 to 5 carbon atoms. Aryl groups are preferably carbocyclic or heterocyclic systems containing one or two aromatic rings (when two rings are present, they are preferably fused). Examples of suitable aryl rings include benzene, furan, thiophene, pyrrole, pyrazole, triazole, isoxazole, oxazole, thiazole, isothiazole, pyran, pyrone, dioxin, pyridine, pyridizine, purimidine, pyrazine, triazine, indene, benzofuran, isobenzofuran, benzothiofuran, indole, napthalene, coumarin, quinoline. and isoquinoline. Simple aryl groups such as phenyl, napthyl, and single-ring heterocycles are preferred. Phenyl is particularly preferred. Preferred aryl lower alkyl groups are those in which the aryl and lower alkyl substituents have the previously defined meanings above. In all cases, normal substituents found on alkyl groups and aromatic rings, such as halogen, amino, hydroxy, and amido groups, can be present and are included within the meaning of alkyl and aryl. However, aryl groups substituted only with hydrogens are preferred.

Certain compounds of the formula indicated above are preferred. When the compound is a succinimide, R is preferably hydrogen or methyl, R₁ is preferably hydrogen or methyl, and R₂ is preferably methyl, ethyl, or phenyl. Especially preferred are 2-methyl-2-ethyl succinimide (ethosuximide), N-methyl-2-phenyl succinimide, and N,2-dimethyl-2-phenyl succinimide (methsuximide).

When the compound is an oxazolidinedione, R is preferably hydrogen or methyl (especially methyl), R₁ is preferably methyl, and R₂ is preferably methyl or ethyl. The compounds N,5,5-trimethyloxazolidinedione (trimethadione) and N,5-dimethyl-5-ethyloxazolidinedione (paramethadione) are especially preferred.

Other suitable anticonvulsant compounds include, but are not limited to, valproic acid, divalproate sodium, phenyloin, phenyloin sodium, clonazepam, primidone, phenobarbitol, phenobarbitol sodium, carbamazepine, amobarbital sodium, methsuximide, metharbital, mephobarbital, mephenyloin, phensuximide, paramethadione, ethotoin, phenacemide, secobarbitol sodium, and clorazepate dipotassium.

The mammal in need thereof in the embodiment for treating CPS according to the present invention is any mammal suffering from central pain syndrome. Preferably, the mammal is a human, but other mammals including, but not limited to, rat, mouse, rabbits, cats, dogs, primates, etc., are intended to be encompassed by the term “mammal”.

Another embodiment or aspect of the present invention is a method for inducing centrally generated pain responses as an animal model for human central pain syndrome. This method involves unilaterally or bilaterally transecting the spinothalamic tract of a non-human mammal at the level of the thoracic/lumbar border, which severs the ascending pathways conveying somatosensory, thermal and nociceptive information to the cortex via the thalamus, to induce centrally generated pain responses in the non-human mammal. The dura is opened to expose the spinal cord and the spinal cord is lifted and rotated. The ventral lateral pain pathways are then severed in the spinal cord to generate a lesion. The lesion is packed with sterile packing material before closing and suturing the overlying muscle and skin.

For the purposes of this embodiment (inducing centrally generated pain responses in a non-human mammal as an animal model for central pain syndrome), the non-human mammal is preferably a rat but can be any non-human mammal disclosed above with regard to the embodiment for treating CPS.

Using the embodiment for inducing centrally generated pain responses in a non-human mammal, thalamic relay cells can be obtained, i.e., as a thalamic brain slice, from an animal that has had a lesion in the spinothalamic pathway and used in an assay to screen and identify a compound that inhibits T-type calcium channels. This assay method involves detecting abnormal thalamic cell excitability (i.e., by extracellular recordings described in Examples 2 and 5) in thalamic relay cells in the absence of the candidate compound as a control. The same thalamic relay cells are contacted with a candidate compound and thalamic cell excitability in the presence of the candidate compound is detected. The candidate compound is identified as a compound that inhibits T-type calcium channels and/or is useful in treating central pain syndrome if thalamic cell excitability is reduced in the presence of the candidate compound. A compound identified to inhibit T-type calcium channels can be further tested in the above described animal model for CPS by conducting behavioral tests for allodynia and hyperalgesia (symptoms of CPS), such as the von Frey's test and the paw withdrawal latency test described in Example 5 below.

In the CPS treatment embodiment, an effective amount of a thalamic anticonvulsant compound to be administered to a mammal suffering from CPS is a pain-reducing amount that alleviates central pain or the perception or sensation of pain. One or more thalamic anticonvulsant compounds may be formulated in a pharmaceutical composition for administration to the subject in need thereof. Such a pharmaceutical composition contains a physiologically/pharmaceutically acceptable carrier, excipient, diluent or auxiliary agent and may contain other compounds, which may be biologically active or inactive.

While any suitable carrier, excipient or diluent known to those of ordinary skill in the art may be employed in the pharmaceutical compositions containing one or more thalamic anticonvulsant compounds, the preferred carrier, excipient or diluent depends upon the preferred mode of administration. Compositions may be formulated for any appropriate mode of administration, including for example, topical, oral, nasal, rectal, intravenous, intracranial, spinal tap, intraperitoneal, transdermal, subcutaneous or intramuscular administration. For parenteral administration, such as by subcutaneous injection, the carrier preferably comprises water, saline, glycerin, propylene glycol, alcohol, a fat, a wax and/or a buffer. For oral administration, any of the above carriers, or a solid carrier such as mannitol, lactose, starch, magnesium stearate, sodium lauryl sulfate, lactose, sodium citrate, calcium carbonate, calcium phosphate, silicates, polyethylene glycol, sodium saccharine, talcum, cellulose, glucose, sucrose, dyes, and magnesium carbonate, may be employed. For rectal administration, an aqueous gel formulation, or other suitable formulations that are well known in the art may be administered. Solid, compositions may also be employed as fillers in soft and hard filled gelatin capsules. Preferred materials for this include lactose or mild sugar and high molecular weight polyethylene glycols. When aqueous suspensions or elixirs are desired for oral administration, the essential active ingredient therein may be combined with various sweetening, or flavoring agents, coloring matter or dyes and, if desired, emulsifying or suspending agents, together with diluents such as water, ethanol, propylene glycol, glycerin and combinations thereof.

Compositions containing one or more thalamic anticonvulsant compounds as an active ingredient may be administered as part of a sustained release formulation. Such formulations may generally be prepared using well known technology and administered by, for example, oral, rectal or transdermal, delivery systems, or by implantation of a formulation or therapeutic device at one or more desired target site(s). Sustained-release formulations may contain a treatment composition comprising a thalamic anticonvulsant compound, dispersed in a carrier matrix and/or contained within a reservoir surrounded by a rate controlling membrane. Carriers for use within such formulations are biocompatible, and may also be biodegradable. The sustained release formulation may provide a relatively constant level of active composition release. The sustained release formulation may be contained in a device that may be actuated by the subject or medical personnel, upon onset of certain symptoms, for example, to deliver predetermined dosages of the treatment composition. The amount of the treatment composition contained within a sustained release formulation depends upon the site of implantation, the rate and expected duration of release.

Administration can thus be by any technique designed to cause the active agent to enter the blood stream of the mammal and be circulated to the appropriate brain tissues. Oral administration is particularly preferred and is known for all of the specific classes of anticonvulsants compounds disclosed above. Particular blood levels desired for optimum performance will vary depending on the type of anticonvulsant compound used and can be determined by direct serum measurements and by the effect of the compound in reducing pain. The dose of anticonvulsant compound administered can range from about 0.1 mg/kg to about 50 mg/kg. Typical initial doses for succinimides range from about 1 to 25 mg/kg, preferably about 2 to about 20 mg of compound per kilogram of mammal. Preferred daily totals are 250 to 1500 mg for ethosuximide (typical adult dosage is 500 mg per day divided into two doses with possible increase over a few weeks to 500-1500 mg per day in 2 or 3 divided doses) and 150 to 1200 mg for methsuximide. For known compounds that have previously been used to treat other disorders, such as epilepsies, initial concentrations can be those concentrations normally used for the other treatment. Adjustments can then be made, upward or downward, depending on the reduction of pain, the measured serum level, and/or side effects that occur. Considerable variation between treated individuals is likely to occur so that the actual dosage used is best obtained by following the pharmacologic effects on the individual being treated. Dosage information is available from numerous sources, such as Goodman and Gillman's The Pharmacological Basis of Therapeutics, 7th Edition, Gillman et al., Eds., McMillan Publishing Company, New York, 1985. Chapter 20 of Goodman and Gillman is specifically directed to drugs effective in the therapy of the epilepsies and includes discussion of a number of the classes of compounds described in this specification. See also, Remington's Manual for information on formulation, dosage, and the like.

Having now generally described the invention, the same will be more readily understood through reference to the following examples which are provided by way of illustration and are not intended to be limiting of the present invention.

### EXAMPLE 1

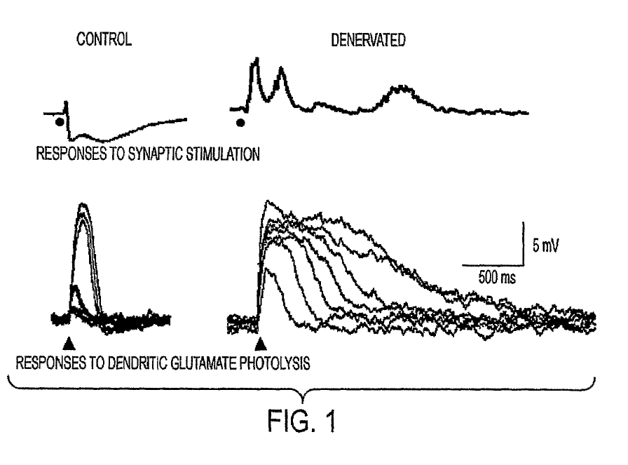

It may be expected based on previous studies in the laboratory of the present inventor that partial denervation and removal of the majority of the excitatory input to area CA1 would result in a quiescent population of cells. In fact, local stimulation within area CA1 triggers prolonged, abnormally oscillatory synaptic responses in lesioned cultures that are never seen in normally innervated tissue (FIG. 1, top row), but only with a delay of 7 or more days after the injury (Wei et al., 2003). The reason is not that denervation increases glutamate receptor expression in CA1 cells, akin to the well known phenomenon of denervation suprasensitivity in muscle. To confirm, the laboratory of the present inventor used photolysis of caged glutamate to compare the responses of CA1 cell dendrites in lesioned and unlesioned cultures.

In control cultures, distal, terminal dendritic branches of CA1 cells respond to weak photolysis of glutamate with passive subthreshold depolarizations. Stronger glutamate applications, however, trigger all-or-none Ca²⁺-sensitive responses that are called plateau potentials (Wei et al., 2001). Calcium imaging reveals that the subthreshold responses generate only small local Ca²⁺ transients due to local NMDA receptor-mediated influx. Plateau potentials, in contrast, are accompanied by Ca²⁺ signals that can be seen throughout the stimulated dendrite within milliseconds, indicative of their rapid electrical propagation along the entire stimulated branch. Intracellular dialysis of the Ca²⁺ chelator BAPTA, or extracellular application of apamin or high concentrations of TEA each was shown to prolong plateau potentials 2-4 fold in control cells, demonstrating that SK-type Ca²⁺-activated K⁺ channels are normally responsible for their termination (Cai et al., 2004). These results demonstrate that terminal dendrites normally respond to strong stimuli with an active spike, mediated by voltage-gated Ca²⁺ channels, that is confined to a single dendritic compartment.

In cultures that have been denervated 7 or more days earlier, CA1 cells display no change in the amplitude or kinetics of the responses to the smallest glutamate pulses, indicating that transmitter supersensitivity does not occur in this model. In striking contrast to control cultures, however, we observe a marked prolongation of plateau potentials was observed in deafferented cells (FIG. 1, bottom row). In deafferented cells, the duration of plateau potentials is increased 8-fold compared to control cultures. These results clearly favor an increase in ‘postsynaptic’ intrinsic excitability over an increase in ‘presynaptic’ network excitability. Prolongation of plateau potentials produced by apamin, TEA, and BAPTA in control CA1 cells have been observed to be all occluded in denervated CA1 cells. It is suggested, therefore, that the prolongation of plateau potentials after partial deafferentation is due, at least in part, to down regulation of the SK-type Ca²⁺ activated K⁺ channels that normally mediate repolarization of the plateau potential. Interestingly, chronic application of NMDAR and non-NMDAR antagonists produces an identical prolongation of plateau potentials, suggesting that it is the decrease in glutamate-driven excitation that triggers this increase in intrinsic excitability.

Prolongation of plateau potentials in denervated CA1 cells is accompanied by a marked facilitation of the ability of dendritic excitation to trigger action potential discharge. Whereas photolysis of caged glutamate on terminal dendrites never elicits action potential discharge in control cells (Cai et al., 2004), the prolonged plateau potentials always elicit trains of action potentials in denervated cells. This data teaches that increased postsynaptic intrinsic excitability may contribute to lesion-induced hyperexcitability. It is further suggested that CA1 cells respond to a decrease in synaptic excitation after denervation with what should have been an adaptive, homeostatic response, namely an amplification of remaining incoming excitatory inputs, and that posttraumatic epilepsy is an ‘unintended’ consequence of this response.

The experiments described below in Examples 2-5 are being pursued because of the several notable parallels between posttraumatic epilepsy and CPS. The present inventor predicted that these two syndromes share common underlying pathophysiological mechanisms: both are associated with brain injuries that produce partial denervation, both develop with a delay after injury, and both are characterized by excessively bursting neurons, where a common mechanism underlying both syndromes is a denervation-induced increase in intrinsic or network excitability, with CPS and posttraumatic epilepsy the maladaptive results of an adaptive, homeostatic response.

### EXAMPLE 2

### Comparing the Excitability of the VB in Ex Vivo Thalamic Brain Slices from Rats in which Ascending Spinal Sensory Pathways have Been Lesioned with Slices from Sham Operated Control Rats

The present inventor hypothesizes that thalamic relay cells in VB respond homeostatically to the decrease in afferent activation with a delayed increase in their excitability, apparent electrophysiologically as prolonged burst responses. This hypothesis will be tested by preparing ex vivo thalamic slices containing the VB from sham operated control rats and rats lesioned 1, 3, 7, 14, and 28 days earlier.

The sites of denervation in the thalamus will first be determined by performing classical silver stains for degenerating axons. Rats will be perfused with paraformaldehyde at 2, 3, and 5 days after the lesions, and cryostat sections will be cut through the thalamus at 15 μm in thickness. A modified version of the Gallyas silver stain will then be used to reveal the degenerating terminals of the lesioned ascending pathways. The present inventor predicts that hyperexditability will occur in or near these sites.

Extracellular recordings will be made at various positions in the VB and surrounding regions corresponding to the areas of most intense silver staining. Field potentials evoked with stimulation in the internal capsule or with local intranuclear stimulation will be compared in the lesioned and control slices. The amplitude of the evoked field potentials (low pass filtering) and number of action potentials (high pass filtering) will be determined over a range of stimulation intensities; so-called input-output functions. Responses to stimulus trains of fixed intensity and number of pulses, delivered at various frequencies, will also be compared. When enhanced bursting is observed, responses will also be elicited in unrelated sensory nuclei, such as the medial or lateral geniculate nuclei, to provide an important control for the health of the slices.

It is predicted by the present inventor that bursts of action potentials will be elicited in denervated VB cells, which will be apparent as field potentials with multiple spike components (e.g., von Krosigk et al., 1993; Ulrich and Huguenard, 1995; D'Arcangelo et al., 2002), in slices from animals with spinal lesions, but not in the slices from sham operated controls. Alternatively, denervated slices may display a lower threshold for burst responses. Human CPS develops with a delay of weeks to months. These experiments would allow determination of the time course with which hyperexcitability develops in this model and identification of the spatial relations between the degeneration of the lesioned ascending pathways and the hyperexcitability.

Should significant differences fail to be detected in evoked responses in denervated and normally innervated cells in control saline, the experiments would be repeated in the presence of muscarinic acetylcholine, adenosine A1, or GABA_{B}R antagonists because these substances block the effects of endogenously released transmitters and are known to promote thereby bursting behavior in thalamic relay cells (McCormick, 1989; von Krosigk et al. 1993; Ulrich and Huguenard, 1995). The present inventor predicts that the latent bursting uncovered by such manipulations will be stronger and more prolonged in the lesioned slices. In addition, a failure to detect hyperexcitability might indicate that the extent of the denervation was insufficient to induce plasticity, both in terms of the fraction of lesioned inputs and the fraction of the VB affected. Should there be failure to detect excessive bursting, the lesions will be performed at a more rostral level so as to interrupt a larger portion of the ascending nociceptive pathways. Alternatively, the lesion of both dorsal column and ventrolateral pathways may produce too large of a denervation in the thalamus or leave too few remaining afferents to trigger excessive, bursting responses from the denervated thalamic cells. Should there be a failure to detect significant hyperexcitability in the denervated thalamus, animals would then be prepared with bilateral lesions of only one pathway (dorsal column or ventrolateral) or with unilateral lesions of both pathways.

### Materials and Methods

Spinal cord lesions. Male Sprague-Dawley rats (3-5 weeks after birth) will be anesthetized with ketamine/xylazine (60/5 mg/kg, i.p.). A T8 laminectomy will be performed using sterile techniques in a laminar flow hood under a dissecting microscope. The dura will be opened with the tip of a 21 g. syringe needle to expose the spinal cord. The dorsal columns will then be transected bilaterally using ophthalmic scissors (Neumann and Woolf, 1999). Care will be taken not to damage the underlying corticospinal tracts. The spinal cord will then be lifted and rotated using a fine, sterile glass rod, and the ventral lateral pain pathways will be severed bilaterally with the ophthalmic scissors. The lesion will be packed with a small piece of sterile gelfoam. The overlying muscles and skin will then be closed and sutured, and the animal will be allowed to recover on a 37° C. heating pad. Sham operated controls will undergo a laminectomy without dural penetration or spinal injury.

Electrophysiology. Experiments were performed on thalamic brain slices from rats which have undergone spinal transection 2-4 weeks previous. Whole cell voltage clamp and extracellular field potential recordings were used to confirm low threshold calcium spikes or inhibitory synaptic potentials mediated by GABA are altered in thalamic relay cells after spinal cord injury.

At various times after introduction of the lesion, animals are anesthetized and decapitated, the brain is removed, and sagittal thalamic slices are cut on a vibratome at a thickness of 350-450 μm. Slices are placed in a moist, well oxygenated holding chamber for >1 hr in physiological saline, consisting of 124 mM NaCl, 3 mM KCl, 1.25 mM NaH₂PO₄, 1.5 mM MgCl₂, 2 mM CaCl₂, 26 mM NaHCO₃, and 10 mM glucose. For recording, slices are placed in a submerged recording chamber (for field potential recording) or on the stage of an up-right microscope (for whole-cell recording) and perfused with oxygenated physiological saline. Electrical stimuli (100 μs in duration, 0.2-10 V in intensity) are delivered through a bipolar electrode located in the internal capsule, just lateral to the ventrobasal complex and the nRT, activating both thalamocortical and corticothalamic fibers, or within the nRT. Extracellular recordings are made with patch pipettes containing physiological saline using standard techniques (e.g., D'Arcangelo et al., 2002). Whole-cell current-clamp recordings, performed as described previously (e.g., Cai et al., 2004), are made from the cell bodies of visually identified thalamic relay cells using patch pipettes with tip resistances of 2-4 MΩ. For study of K⁺ and H− currents, whole-cell pipettes contain: 135 mM K-methanesulphate, 10 mM HEPES, 10 mM NaCl, 1 mM MgCl₂ 1, 0.1 mM K₄BAPTA 0.1, 2 mM Mg²⁺-ATP, and 10 mM phosphocreatine (adjusted to pH 7.3 with KOH). All experiments are performed at room temperature. For analysis of T-type Ca²⁺ currents, Cs-methanesulphate replaces K-methanesulphate so as to block outward K⁺ currents. For analysis of inhibitory synaptic currents (Example 4), KCl replaces K-methanesulphate so as to increase the driving force through the GABA_{A}R mediated Cl⁻ channels. For study of glutamatergic excitatory currents, the tip of the pipette contains saline in which CsF replaces K-methanesulphate so as to block GABA_{A}R-mediated currents in just the recorded cell, without affecting network excitability (e.g., Brager et al., 2002). Electrophysiological data is amplified, and then digitized and analyzed post hoc using a Digidata analog/digital converter and pClamp9 software (Axon Instruments). Cell capacitance and series resistance (reported in Capogna et al. (1997) to average 10.0±0.5 MΩ for 50 cells) are monitored regularly and compensated. In voltage-clamp recordings of T-type Ca²⁺ currents, 50% increases in current amplitude that occur with voltage steps of 5 mV are considered evidence of voltage escape, and the data is not further analyzed (Thompson and Wong, 1991). Methods include with whole-cell voltage- and current-clamp recording and its application for the analysis of T-type Ca²⁺ current (Thompson and Wong, 1991), paired-pulse ratios (Debanne et al., 1996), and evoked and miniature IPSCs and EPSCs (Capogna et al., 1995; 1997).

Spontaneous mIPSCs and mEPSCs are acquired and analyzed using whole-cell recording techniques, as described previously (Capogna et al., 1997) and employed extensively in the laboratory of the present inventor. Cells are whole-cell voltage-clamped at −75 mV. mEPSCs are recorded in the presence of 0.5 μM tetrodotoxin and 40 μM bicuculline methochloride, to block action potentials and GABA_{A}RS. mIPSCs are recorded in the presence of 0.5 μM tetrodotoxin, and 40 μM DNQX and 40 μM AP5 to block ionotropic glutamate receptors. Membrane current are recorded continuously for 5 min. Data are analyzed using the pClamp software. The amplitudes, rise and decay times, and inter-event intervals of mIPSCs and mEPSCs are compared using the Kolmogorov-Smirnov test, which estimates the probability that the two distributions are not significantly different.

Anatomy. In all experiments, the spinal cord is removed and fixed after the thalamic slices have been prepared in order to verify the site and extent of the lesion. Sections are cut some time later on the vibratome, mounted onto pre-coated microscope slides, and stained for Nissl substance using cresyl violet. The lesions are compared with the electrophysiological results post-hoc. Lesions not significantly involving the spinothalmic tract and the lateral pain pathways are predicted not to result in substantial alterations in the electrophysiological behavior of the thalamic relay cells. The silver impregnation method for degenerating fibers that is used employ is a modified version of the methods of Gallyas et al. (1980), as described in detail by Nadler and Evenson (1983).

### EXAMPLE 3

### Comparing the Intrinsic Excitability of Thalamic Relay Neurons in the VB in Control and Partially Denervated Thalamic Brain Slices

The present inventor predicts that hyperexcitability in denervated VB cells may result from increased intrinsic excitability. T-type Ca²⁺ currents in thalamic relay cells have been shown to be affected by injury (Chung et al., 1993) and an increase in T-type or H-type currents, or a decrease in a K⁺ current, could also produce increased bursting. This hypothesis will be tested by using straightforward whole-cell current- and voltage-clamp recording from VB relay cells in thalamic slices taken at the optimum time and place after spinal lesion, as determined in the experiments of Example 2, and compared to data from slices taken from sham operated control rats. Recordings will be performed in extracellular saline containing glutamate and GABA receptor antagonists so that intrinsic neuronal excitability can be studied in the absence of synaptic influences. The basic electrophysiological properties of control and denervated relay cells will first be compared. The resting membrane potentials of the cells will be noted, and the neuronal input resistance will be assessed, in voltage-clamp mode, with 5 mV hyperpolarizing voltage steps from the resting potential. Next, the cell's potential will be adjusted to −80 mV with current injection and compare, in current-clamp mode, the number and frequency of action potentials elicited with depolarizing current pulses (500 ms duration) of varying amplitudes in control and lesioned slices. Bursts of action potentials are elicited only from hyperpolarized voltages because they are mediated by T-type voltage-dependent Ca²⁺ channels that are inactivated at depolarized voltages (McCormick, 1989). Basic action potential parameters (threshold, amplitude, duration) will also be compared. In a third set of experiments, the amplitude, duration, and kinetics of T-type Ca²⁺ current underlying burst discharges will be recorded directly in voltage-clamp mode in the presence of TTX to block fast action potentials using pipettes with a Cs⁺-based saline to block K⁺ currents. T-type currents will first be elicited in control and lesioned cells with depolarizing steps of varying amplitude applied from a range of holding potentials so as to determine the voltage-dependence of activation. T-type Ca²⁺ currents are characterized by strong voltage-dependent inactivation and recovery from inactivation (Coulter et al., 1989). These parameters will be quantified by clamping the membrane potential to a depolarized pre-potential (−55 mV) to inactivate the channels. Currents will be recorded at a fixed test potential (−50 mV) while preceding the test step with hyperpolarizing voltage steps of varying amplitude and duration to remove inactivation.

These experiments will be followed with comparable analyses of H-current in VB relay cells in control and lesioned slices. Experiments will be performed in saline containing TTX and glutamate and GABA_{A}R antagonists. Whole-cell pipettes will contain a K-methanesulphate-based solution. Serotonin and norepinephrine increase cAMP and thereby the voltage-dependence of H-current activation to more depolarized levels (Pape and McCormick, 1989; Lüthi and McCormick, 1999). Voltage-dependence of H-current activation will be determined by clamping the cell's membrane potential to −50 mV and delivering hyperpolarizing activation steps of 4 s duration to various potentials between −100 and −55 mV, followed by a step back to −50 mV. H-current amplitude will be measured by the amplitude of the tail current at −50 mV (after subtracting leak currents) and plotted as a function of the activation step voltage.

Transient and sustained K⁺ currents will be quantified in VB relay cells in control and lesioned slices in saline containing TTX, and glutamate and GABA_{A}R antagonists. Whole-cell pipettes will contain a K-methanesulphate-based pipette solution. Cells will be voltage-clamped to −90 mV to remove inactivation and depolarizing test steps of 4 s duration to various potentials between −90 and 0 mV will be delivered, thus activating both transient and sustained currents. The amplitude of the sustained current will be quantified as the leak subtracted current amplitude at the end of the 4 s activation step, and the transient current amplitude will be quantified as the difference between the peak current early in the test step and the sustained current at the end of the pulse.

At the conclusion of the experiments in this Example, a thorough biophysical description of T-type Ca²⁺ current, H-current, and K⁺ currents in control and lesioned thalamic relay cells, as well as the effectiveness with which these currents can generate low threshold action potential bursts, will have been obtained.

The hypothesis here predicts that an increase in excitability after partial denervation should be apparent as an increase in the number of action potentials in each burst, an increase in the amplitude and/or duration of the underlying burst envelope, and/or a shift in the threshold voltage for eliciting bursts. One explanation for these outcomes is a change in the amplitude, kinetics, or voltage-dependence of the T-type Ca²⁺ current, H-current, or K⁺ currents underlying and shaping these bursts.

### EXAMPLE 4

### Comparing the Strength of GADAergic Synaptic Inhibition and Glutamatergic Excitation in Thalamic Relay Neurons in the VB in Control and Partially Denervated Thalamic Brain Slices

It has been postulated that GABAergic inhibition in the thalamus may be altered in CPS (Ralston et al., 1996; Canavero and Bonicalzi, 1998). Hyperpolarizing IPSPs in thalamic relay cells are critical determinants of burst firing because they relieve the voltage-dependent inactivation of the T-type Ca²⁺ currents underlying bursting. One mechanism to account for hyperexcitability in the denervated thalamus is therefore that a change in the strength of GABAergic inhibition (as opposed to the change in intrinsic excitability tested in Example 3). Alternatively, changes in glutamatergic excitation in the spinal cord and brainstem underlie other chronic pain syndromes (Willlis, 2002; Ji et al., 2003) and could also affect bursting.

These hypotheses will be tested using whole-cell current- and voltage-clamp recordings from relay cells in thalamic slices taken at the optimum time after spinal lesion, as determined in the experiments described in Aim 1, and compared to data from slices taken from sham operated control rats. Experiments will first be performed in extracellular saline containing glutamate receptor antagonists so that GABAergic inhibitory postsynaptic currents (IPSCs) can be studied in isolation. IPSCs will be evoked with extracellular stimulation within the adjacent nRT. First, the amplitude of IPSCs in relay cells will be recorded over a range of stimulation intensities. The pre- and postsynaptic properties of these inhibitory synapses will then be compared. The paired-pulse ratio of evoked IPSCs will be determined with pairs of stimuli delivered at varying interstimulus intervals from 20-500 ms. The paired pulse ratio is a standard electrophysiological indicator of presynaptic release probability (Ulrich and Huguenard, 1995). Next, miniature IPSCs (mIPSCs) in the presence of TTX will be recorded. Cumulative probability distributions will be made of mIPSC amplitude and frequency. Because mIPSCs result from the spontaneous fusion of single GABA containing presynaptic vesicles, mIPSC amplitude distributions provide an indicator of postsynaptic GABA_{A}R sensitivity, whereas mIPSC frequency is positively correlated with presynaptic release probability (Capogna et al., 1995). The rising and falling phases of averaged mIPSCs will be fit with exponentials in order to compare the kinetics of the responses. Finally, inhibitory postsynaptic potentials (IPSPs) of various amplitudes will be elicited in current-clamp mode at several holding potentials in order to test the ability of IPSCs to trigger rebound low threshold Ca²⁺ spike bursts (von Krosigk et al., 1993).

Standard electrophysiological techniques will also be used to examine whether there has been any change in behavior of remaining, non-lesioned excitatory inputs to relay cells, as has been suggested to occur following similar lesions in cats (Koyama et al., 1993). VB relay cells in control and lesioned slices will be recorded with current flow through GABA_{A}Rs in the recorded cell blocked by a CsF-based pipette solution (Brager et al., 2002). An attempt will be made to activate a high percentage of corticothalamic afferents with stimulation in the external capsule and then other afferent pathways with local stimulation within VB. The amplitude of the synaptic currents will be compared at −80 mV and +40 mV to derive a ratio of AMPAR- to NMDAR-mediated current (e.g. Abegg et al., 2004). Pairs of stimuli will be delivered at −80 mV at various interstimulus intervals (20-500 ms) for calculation of the paired pulse ratio of the pathways; a well established surrogate indicator of release probability (e.g. Debanne et al., 1996). Finally, mEPSCs will be recorded in the presence of TTX to provide a measure of synaptic AMPAR expression levels (mEPSC amplitude) and an independent measure of release probability (mEPSC frequency).

At the conclusion of the experiments in this Example, the strength of GABAergic inhibition and glutamatergic excitation in thalamic relay cells will have been quantified and it will have been determined whether they are altered by chronic deafferentation.

The present inventor predicts that an increase in excitability after partial denervation may be accounted for by a change in GABA_{A}R-mediated inhibition or glutamatergic excitation in relay cells. Somewhat counterintuitively, an increase inhibition would be most effective in promoting bursting (Kim et al., 1997). If a change in inhibition is observed, then it will be of interest to look at the synaptic excitation and intrinsic excitability of nRT interneurons, as well as their feedback inhibition, in order to determine the cause of the increased inhibition in relay cells.

Under voltage-clamp, the current-voltage relationship, maximal current density, threshold, inactivation rate, and voltage dependence of recovery from inactivation of Ca²⁺ currents, under pharmacological conditions in which Na⁺ and K⁺ currents are blocked, were measured. GABAergic inhibitory synaptic transmission in thalamic relay cells were studied in separate experiments in which excitatory amino acid receptors were blocked pharmacologically. Determination of the amplitude of inhibitory synaptic currents under voltage-clamp in response to electrical stimulation within the nucleus reticularis thalami was also observed. Stimulus-response curves were obtained and the kinetics of the resulting inhibitory synaptic currents were determined. Under current-clamp conditions, the relationship between the amplitude of nRT evoked inhibitory synaptic potentials and the amplitude and integral of resulting rebound, low threshold calcium spikes was identified. Low threshold Ca²⁺ spikes were directly (though changes in channel expression levels) or indirectly (through potentiation of inhibitory synaptic responses, for example) amplified in cells in slices from lesioned animals as compared to cells in slices from uninjured control animals.

### EXAMPLE 5

### Comparing the Effects of ‘Thalamic Anticonvulsants’ on the Excitability of Thalamic Relay Neurons in the VB in Control and Partially Denervated Thalamic Brain Slices

The rationale for the experiments in this example is that if enhanced bursting in thalamocortical relay cells underlies the pathology of CPS, then drugs that inhibit thalamic bursting in human absence epilepsy would offer sorely needed therapeutic relief. The amplitude and duration of abnormal field potentials in denervated thalamic slices can be reduced to control levels with ethosuximide and other thalamic anti-convulsants is tested by using whole-cell current- and voltage-clamp recording from relay cells in thalamic slices taken at the optimum time and place after spinal lesion, as determined in the experiments of Example 2, and compared to data from slices taken from sham operated control rats. Recordings are performed in control extracellular saline. First, field potentials are evoked with stimulation in the internal capsule or with local stimulation, as in Example 2, before and after application of the T-channel blocker, ethosuximide, at clinically relevant concentrations (20 to 140 μg/ml) (Coulter et al., 1990). The non-anticonvulsant succinimide parent ring structure serves as a negative control. Similar experiments will be performed with methylphenylsuccinimide (5 mM), valproic acid (0.1-1 mM), and U-92032 (10 μM), another specific inhibitor of T-type Ca²⁺ current (Porcello et al., 2003), all at clinically relevant concentrations. Any drug displaying an ability to inhibit bursting in lesioned slices can be tested further for its ability to inhibit T-type Ca²⁺ current-mediated action potential bursts in whole-cell current-clamp recordings, as in Example 3.

These drugs should be effective regardless of the cause of the excessive thalamic bursting. Nevertheless, if evidence of increased inhibition is revealed in Example 4 of GABAgergic synaptic inhibition, it will be of interest to determine whether reversing the increased inhibition in lesioned slices will be effective in inhibiting excessive thalamic bursting and hyperexcitability. To this aim, submaximal concentrations of the GABA_{A}R antagonist bicuculline methochloride (0.5-5 μM) or the GABA_{A}R benzodiazepine site inverse agonist, methyl-6,7-dimethoxy-4-ethyl-beta-carboline-3-carboxylate (DMCM, 1-10 μM) will be applied. While these later drugs are unlikely to be of therapeutic utility because of their widespread non-thalamic effects, these experiments will nonetheless provide important insights into the genesis of the excessive thalamic bursting.

### Materials and Methods

Spinothalamic tract transection. The spinothalamic tract is transected bilaterally at the level of the thoracic/lumbar border, thereby severing the ascending pathways conveying somatosensory, thermal and nociceptive information to the cortex via the thalamus. Male, adult Sprague-Dawley rats (ca. 200 gm) are anesthetized with pentobarbital and a T8 laminectomy is performed. The dura is opened with the tip of a 21 g. syringe needle to expose the spinal cord. The spinal cord is then lifted and rotated using a fine, sterile glass rod, and the ventral lateral pain pathways are severed electrolytically using a 1 MΩ sharpened tungsten needle and a 45 V pulse for 1 min. The lesion is packed with a small piece of sterile gelfoam. The overlying muscles and skin are closed and sutured, and the animal is allowed to recover on a 37° C. heating pad. Sham-operated controls undergo a laminectomy without dural penetration or spinal injury. This spinal transection procedure is a useful model of central pain syndrome where increased pain sensitivity and allodynia were observed. At various times after the lesion, standard behavioral tests were completed to assay the threshold for a stimulus to be perceived as painful, and allodynia was performed on awake, behaving animals. These procedures have been fully approved by the University of Maryland School of Medicine Animal Care and Use Committee.

Electrophysiology. At various times after the lesion, animals are anesthetized and decapitated, the brain is removed, and thalamic slices are cut on a vibratome at a thickness of 400-500 μm. Slices are placed in a moist, well oxygenated holding chamber for >1 hr in physiological saline, consisting of (in mM) 124 NaCl, 3 KCl, 1.25 NaH₂PO₄, 1.5 MgCl₂, 2 CaCl₂, 26 NaHCO₃, and 10 glucose. For recording, slices are placed in a submerged recording chamber and perfused with oxygenated physiological saline at room temperature. Electrical stimuli (100 μs in duration, 0.2-10 V in intensity) are delivered through a bipolar electrode located in the internal capsule, just medial to the ventrobasal complex and the nRT, activating both thalamocortical and corticothalamic fibers. Extracellular recordings are made with patch pipettes containing physiological saline using standard techniques (e.g. D'Arcangelo et al., 2002). Responses are amplified 100× (NPI Electronics) and digitized for later off-line analyses (Clampex, Axon Instruments). Drugs (i.e., 700 μM ethosuximide) are added by bath perfusion.

Behavioral tests. The von Frey filament test is used to test for allodynia. Rats are habituated to stand on their hind paws and lean against the experimenter's hand. Mechanical stimulation is delivered by a set of calibrated Semmes-Weinstein monofilaments (Von Frey filaments; Stoelting, Wood Dale, Ill.). The bending force of the filaments ranges from 9 mg to 300 g. The starting filament is 2 g (4.31 marking) and a descending series of the filaments is used when the rat responded to the starting filament. Each filament is tested five times at an interval of a few seconds. If the animal withdraws its paw at least two times after pricking with a filament, the rat is considered responsive to that filament. The response threshold is defined as the lowest force of the filaments that produces at least two withdrawal responses in five tests.

The paw withdrawal latency test is used to assess hyperalgesia. Rats are placed on the glass surface of the Paw Thermal Stimulator System (Plantar Analgesia Instrument, Stoelting, Wood Dale, Ill.) under an inverted clear plastic cover and allowed to acclimatize for 15-30 min. A radiant heat stimulus is then applied to the plantar surface of each hind paw or fore paw from underneath the glass floor with a high-intensity projector lamp bulb (50 W). The paw withdrawal latency is automatically recorded when the rat withdraws its paw from the stimulus. The stimulus intensity is adjusted by controlling the bulb voltage to derive an average baseline withdrawal latency of ˜10.0 s in naive animals. A 20 s cutoff is used to prevent tissue damage. Three measures, separated by a 5 min interval, are obtained for each hind paw and fore paw, and the mean is designated the latency. Ethosuximide (500 mg/kg) was administered intraperitoneally in both the von Frey's test for allodynia and the paw withdrawal latency test for hyperalgesia.

### Results

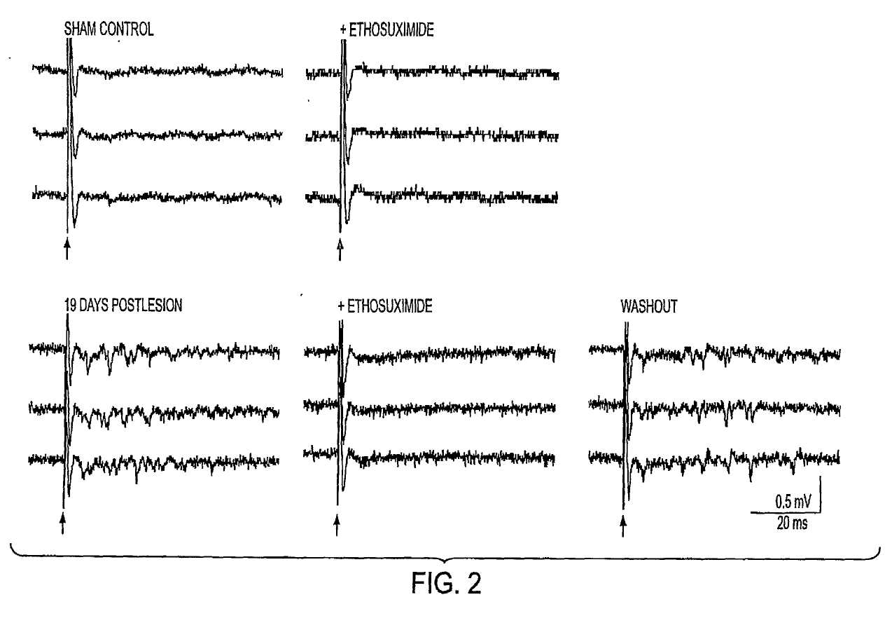

Thalamic bursting was observed in extracellular recordings of neuronal activity in thalamic brain slices from an animal whose spinothalamic tract was lesioned 19 days earlier (FIG. 2, lower row), but not in slices from a sham control animal (FIG. 2, upper row). This thalamic bursting is apparent as a larger number of downward deflections after each stimulus. Furthermore, ethosuximide (700 μM) blocked the bursting activity in the lesioned slices selectively, without affecting the normal evoked responses in control slices.

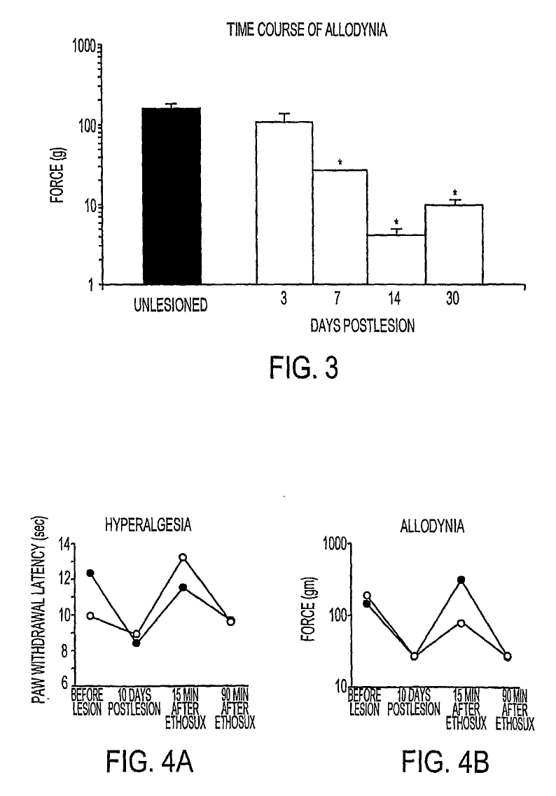

Allodynia, as assayed with the von Frey's test, develops only with a delay after spinothalamic tract transection (FIG. 3). Allodynia, a symptom of human central pain syndrome, refers to an alteration in pain perception such that a normally innocuous stimulus is perceived as painful. In using the von Frey's test, allodynia is indicated by a decrease in the level of force that is needed to trigger the response to paw withdrawal.

Hyperalgesia refers to a decrease in the threshold of a normally painful stimulus that is necessary to elicit a response, and is another symptom of human central pain syndrome. In the assay for hyperalgesia, the latency of foot withdrawal to thermal stimulation is measured, with a decreased latency indicating a decreased tolerance for the weakly painful stimulus. Allodynia (FIG. 4B) and hyperalgesia (FIG. 4A) in animals with lesioned spinothalamic tracts are both reduced transiently upon administration of ethosuximide (500 mg/kg).

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Abegg, M. H., Savic, N., Ehrengruber, M. U., McKinney, R. A., and Gähwiler, B. H. (2004) Epileptiform activity in rat hippocampus strengthens excitatory synapses. J. Physiol. 554:439-448.
Al-Chaer, E. D., Lawand, N. B., Westlund, K. N., and Willis, W. D. (1996) Visceral nociceptive input into the ventral posterolateral nucleus of the thalamus: a new function for the dorsal column pathway. J. Neurophysiol. 76:2661-2674.
Alexander, G. M., and Godwin, D. W. (2005) Presynaptic inhibition of corticothalamic feedback by metabotropic glutamate receptors. J. Neurophysiol. 94:163-175.
Bennett, A. D., Chastain, K. M., and Hulsebosch, C. E. (2000a) Alleviation of mechanical and thermal allodynia by CGRP (8-37) in a rodent model of chronic central pain. Pain 86:163-175.
Bennett, A. D., Everhart, A. W., and Hulsebosch, C. E. (2000b) Intrathecal administration of an NMDA or a non-NMDA receptor antagonist reduces mechanical but not thermal allodynia in a rodent model of chronic central pain after spinal cord injury. Brain Res. 859:72-82.
Bernard, C., Anderson, A., Becker, A., Poolos, N. P., Beck, H., and Johnston, D. (2004) Acquired dendritic channelopathy in temporal lobe epilepsy. Science 305:532-535.
Besson, J. M., and Chaouch, A. (1987) Peripheral and spinal mechanisms of nociception. Physiol. Rev. 67:67-186.
Bonica, J. J. (1991) Introduction: semantic, epidemiological, and educational issues. In: Pain and central nervous system disease: the central pain syndromes. (Raven Press: New York), pp. 1-11.
Brager, D. H., Capogna, M., and Thompson, S. M. (2002) Short-term synaptic plasticity, simulation of nerve terminal dynamics, and the effects of protein kinase C activation in rat hippocampus. J. Physiol. 541:545-559.
Cai, X., Liang, C. W., Muralidharan, S., Kao, J. P., Tang, C. M., and Thompson, S. M. (2004). Unique roles of SK and Kv4.2 potassium channels in dendritic integration. Neuron 44:351-364.
Canavero, S., and Bonicalzi, V. (1998) The neurochemistry of central pain: evidence from clinical studies, hypothesis and therapeutic implications. Pain 74: 109-114.
Capogna, M., Gähwiler, B. H., and Thompson, S. M. (1995) Presynaptic enhancement of inhibitory synaptic transmission by protein kinases A and C in the rat hippocampus in vitro. J. Neurosci. 15:1249-1260.
Capogna, M., Gähwiler, B. H., and Thompson, S. M. (1997) Ca²⁺ or Sr2+ partially rescues synaptic transmission in hippocampal cultures treated with botulinum toxin A and C, but not tetanus toxin. J. Neurosci., 17:7190-7202.
Casey, K. L. (1991) Pain and central nervous system disease: a summary and overview. In: Pain and central nervous system disease: the central pain syndromes. (Raven Press: New York), pp. 1-11.
Christensen, M. D., Everhart, A. W., Pickelman, J. T., and Hulsebosch, C. E. (1996) Mechanical and thermal allodynia in chronic central pain following spinal cord injury. Pain 68:97-107.
Chung, J. M., Huguenard, J. R., and Prince, D. A. (1993) Transient enhancement of low-threshold calcium current in thalamic relay neurons after corticectomy. J. Neurophysiol. 70: 20-27.
Coulter, D. A., Huguenard, J. R., and Prince, D. A. (1989) Calcium currents in rat thalamocortical relay neurones: kinetic properties of the transient, low-threshold current. J. Physiol. 414:587-604.
Coulter, D. A., Huguenard, J. R., and Prince, D. A. (1990) Differential effects of petit mal anticonvulsants and convulsants on thalamic neurones: calcium current reduction. Br. J. Pharmacol. 100:800-806.
Davis, K. D., Kiss, Z. H. T., Tasker, R. R., and Dostrovsky, J. O. (1996) Thalamic stimulation-evoked sensations in chronic pain patients and in nonpain (movement disorder) patients. J. Neurophysiol. 75:1026-1037.
D'Arcangelo, G., D'Antuono, M., Biagini, G., Warren, R., Tancredi, V., and Avoli, M. (2002) Thalamocortical oscillations in a genetic model of absence seizures. Eur. J. Neurosci. 16:2383-2393.
Debanne, D., Guérineau, N. C., Gähwiler, B. H., and Thompson, S. M. (1996) Paired-pulse facilitation and depression at unitary synapses in rat hippocampus: quantal fluctuation affects subsequent release. J. Physiol. 491:163-176.
Dostrovsky, J. O. (2000) Role of thalamus in pain. Prog. Brain Res. 129:245-257.
Dubner, R., and Ren, K. (2004) Brainstem mechanisms of persistent pain following injury. J. Orofac. Pain. 18:299-305.
Frick, A., Magee, J., and Johnston, D. (2004) LTP is accompanied by an enhanced local excitability of pyramidal neuron dendrites. Nat. Neurosci. 7:126-135.
Gallyas, F., Wolff, J. R., Bottcher, H., and Zaborszky, L. (1980) A reliable and sensitive method to localize terminal degeneration and lysosomes in the central nervous system. Stain Technol. 55:299-306.
Giesler, G. J., Spiel, H. R., and Willis, W. D. (1981) Organization of spinothalamic tract axons within the rat spinal cord. J. Comp. Neurol. 195:243-252.
Golshani, P., Warren, R. A., and Jones, E. G. (1998) Progression of change in NMDA, non-NMDA, and metabotropic glutamate receptor function at the developing corticothalamic synapse. J. Neurophysiol. 80:143-154.
Gonzales, G. R. (1995) Central pain: diagnosis and treatment strategies. Neurology 45:S11-6
Greenspan, J. D., Ohara, S., Sarlani, E., and Lenz, F. A. (2004) Allodynia in patients with post-stroke central pain (CPSP) studied by statistical quantitative sensory testing within individuals. Pain 109:357-366.
Gucer, G., Niedermeyer, E., and Long, D. M. (1978) Thalamic EEG recordings in patients with chronic pain. J. Neurol. 219:47-61.
Guilbaud, G., Peschanski, M., Gautron, M., and Binder, D. (1980) Neurones responding to noxious stimulation in VB complex and caudal adjacent regions in the thalamus of the rat. Pain 8:303-318.
Gutierrez, C., Cox, C. L., Rinzel, J., and Sherman, S. M. (2001) Dynamics of low-threshold spike activation in relay neurons of the cat lateral geniculate nucleus. J. Neurosci. 21:1022-1032.
Hao, J. X., and Xu, X. J. (1996) Treatment of a chronic allodynia-like response in spinally injured rats: effects of systemically administered excitatory amino acid receptor antagonists. Pain 66:279-285.
Huguenard, J. R., and McCormick, D. A. (1992) Simulation of the currents involved in rhythmic oscillations in thalamic relay neurons. J. Neurophysiol. 68:1373-1383.
Huguenard, J. R. (1996) Low-threshold calcium currents in central nervous system neurons. Annu. Rev. Physiol. 58:329-348.
Huguenard, J. R. (2002) Block of T-type Ca2+ channels is an important action of succinimide antiabsence drugs. Epilepsy Curr. 2:49-52.
Hulsebosch, C. E., Xu, G. Y., Perez-Polo, J. R., Westlund, K. N., Taylor, C. P., and McAdoo, D. J. (2000) Rodent model of chronic central pain after spinal cord contusion injury and effects of gabapentin. J. Neurotrauma 17:1205-1217.
Jeanmonod, D., Magnin, M., and Morel, A. (1993) Thalamus and neurogenic pain: physiological, anatomical, and clinical data. Neuroreport 4:475-478.
Ji, R. R., Kohno, T., Moore, K. A., and Woolf, C. J. (2003) Central sensitization and LTP: do pain and memory share similar mechanisms? Trends Neurosci. 26:696-705.
Kajander, K. C., and Giesler, G. J. (1987) Effects of repeated noxious thermal stimuli on the responses of neurons in the lateral cervical nucleus of cats: evidence for an input from A-nociceptors to the spinocervicothalamic pathway. Brain Res. 436:390-395.
Kelly, K. M., Gross, R. A., and Macdonald, R. L. (1990) Valproic acid selectively reduces the low-threshold (T) calcium current in rat nodose neurons. Neurosci. Lett. 116:233-238.
Kim, U., Sanchez-Vives, M. V., and McCormick, D. A. (1997) Functional dynamics of GABAergic inhibition in the thalamus. Science 278:130-134.
Kim, D., Park, D., Choi, S., Lee, S., Sun, M., Kim, C., and Shin, H. S. (2003) Thalamic control of visceral nociception mediated by T-type Ca2+ channels. Science 302:117-119.
Koyama, S., Katayama, Y., Maejima, S., Hirayama, T., Fujii, M., and Tsubokawa, T. (1993) Thalamic neuronal hyperactivity following transection of the spinothalamic tract in the cat: involvement of N-methyl-D-aspartate receptor. Brain Res. 612:345-350.
Lenz, F. A., Tasker, R. R., Dostrovsky, J. O., Kwan, H. C., Gorecki, J., Hirayama, T., and Murphy, J. T. (1987) Abnormal single-unit activity recorded in the somatosensory thalamus of a quadriplegic patient with central pain. Pain 31:225-236.
Lenz, F. A., Kwan, H. C., Dostrovsky, J. O., and Tasker, R. R. (1989) Characteristics of the bursting pattern of action potentials that occurs in the thalamus of patients with central pain. Brain Res. 496:357-360.
Lenz, F. A., Gracety, R. H., Baker, F. H., Richardson, R. T., and Dougherty, P. M. (1998) Reorganization of sensory modalities evoked by microstimulation in region of the thalamic principal sensory nucleus in patients with pain due to nervous system injury. J. Comp. Neurol. 399:125-138.
Lüthi, A., and McCormick, D. A. (1999) Modulation of a pacemaker current through Ca2+-induced stimulation of cAMP production. Nat. Neurosci. 2:634-641.
Ma, W., Peschanski, M., and Besson, J. M. (1986) The overlap of spinothalamic and dorsal column nuclei projections in the ventrobasal complex of the rat thalamus: a double anterograde labeling study using light microscopy analysis. J. Comp. Neurol. 245:531-540.
McCormick, D. A. (1989) Cholinergic and noradrenergic modulation of thalamocortical processing. Trends Neurosci. 12:215-221.
McCormick, D. A., and Bal, T. (1994) Sensory gating mechanisms of the thalamus. Curr. Opin. Neurobiol. 4:550-556.
McCormick, D. A., and Contreras, D. (2001) On the cellular and network bases of epileptic seizures. Annu. Rev. Physiol. 63:815-846.
McKinney, R. A., Debanne, D., Gähwiler, B. H., and Thompson, S. M. (1997) Lesion-induced axonal sprouting and hyperexcitability in the hippocampus in vitro: implications for the genesis of posttraumatic epilepsy. Nature Medicine, 3:990-996.
Nadler, J. V., and Evenson, D. A. (1983) Use of excitatory amino acids to make axon-sparing lesions of hypothalamus. Methods Enzymol. 103:393-400.
Ness, T. J., San Pedro, E. C., Richards, J. S., Kezar, L., Liu, H. G., and Mountz, J. M. (1998) A case of spinal cord injury-related pain with baseline rCBF brain SPECT imaging and beneficial response to gabapentin. Pain 78:139-143.
Neumann, S., and Woolf, C. J. (1999) Regeneration of dorsal column fibers into and beyond the lesion site following adult spinal cord injury. Neuron 23:83-91.
Owens, C. M., Zhang, D., and Willis, W. D. (1992) Changes in the response states of primate spinothalamic tract cells caused by mechanical damage of the skin or activation of descending controls. J. Neurophysiol. 67:1509-1527.
Panyiotopolous, C. P. (1997) Absence epilepsies. In: Epilepsy: a comprehensive textbook. J. Engel and T. A. Pedley, Eds. (Lippincott-Raven Publishers: Philadelphia), pp. 2327-2346.
Pape, H, C., Budde, T., Mager, R., and Kisvarday, Z. F. (1994) Prevention of Ca2+-mediated action potentials in GABAergic local circuit neurones of rat thalamus by a transient K+ current. J. Physiol. 478:403-422.
Peschanski, M., Guilbaud, G., Gautron, M., and Besson, J. M. (1980) Encoding of noxious heat messages in neurons of the ventrobasal thalamic complex of the rat. Brain Res. 197:401-413.
Peschanski, M., and Mantyh, P. W. (1983) Efferent connections of the subfascicular area of the mesodiencephalic junction and its possible involvement in stimulation-produced analgesia. Brain Res. 263:181-190.
Peschanski, M., Kayser, V., and Besson, J. M. (1986) Behavioral evidence for a crossed ascending pathway for pain transmission in the anterolateral quadrant of the rat spinal cord. Brain Res. 376:164-168.
Porcello, D. M., Smith, S. D., and Huguenard, J. R. (2003) Actions of U-92032, a T-type Ca2+ channel antagonist, support a functional linkage between IT and slow intrathalamic rhythms. J. Neurophysiol. 89: 177-185.
Ralston, H. J., Ohara, P. T., Meng, X. W., Wells, J., and Ralston, D. D. (1996) Transneuronal changes of the inhibitory circuitry in the macaque somatosensory thalamus following lesions of the dorsal column nuclei. J. Comp. Neurol. 371: 325-335.
Salt, T. E. (1986) Mediation of thalamic sensory input by both NMDA receptors and non-NMDA receptors. Nature 322:263-265.
Shah, M. M., Anderson, A. E., Leung, V., Lin, X., and Johnston, D. (2004) Seizure-induced plasticity of h channels in entorhinal cortical layer III pyramidal neurons. Neuron 44:495-508.
Steriade, M. (2004) Acetylcholine systems and rhythmic activities during the waking-sleep cycle. Prog. Brain Res. 145:179-196.
Tasker, R. R., de Carvalho, G., and Dostrovsky, J. O. (1991) The history of central pain syndromes, with observations concerning pathophysiology and treatment. In: Pain and central nervous system disease: the central pain syndromes. (Raven Press: New York), pp. 31-58.
Thompson, S. M., and Wong, R. K. S. (1991) Development of calcium current subtypes in isolated rat hippocampal pyramidal cells. J. Physiol. 439:671-689.
Timofeev, I., and Steriade, M. (2004) Neocortical seizures: initiation, development and cessation. Neuroscience 123:299-336.
Triggs, W. J., and Beric, A. (1992) Sensory abnormalities and dysaesthesias in the anterior spinal artery syndrome. Brain 115:189-198.
Ulrich, D., and Huguenard, J. R. (1995) Purinergic inhibition of GABA and glutamate release in the thalamus: implications for thalamic network activity. Neuron 15: 909-918.
Vierck, C. J., Greenspan, J. D., and Ritz, L. A. (1990) Long-term changes in purposive and reflexive responses to nociceptive stimulation following anterolateral chordotomy. J. Neurosci. 10:2077-2095.
von Krosigk, M., Bal, T., and McCormick, D. A. (1993) Cellular mechanisms of a synchronized oscillation in the thalamus. Science 261:361-364.
Waldron, J. N., Ghosh, S., and Zarzecki, P. (1989) Multiple inputs to a population of thalamocortical neurons projecting to cat somatosensory cortex. Exp. Brain Res. 74:105-115.
Weng, H. R., Lenz, F. A., Vierck, C., and Dougherty, P. M. (2003) Physiological changes in primate somatosensory thalamus induced by deafferentation are dependent on the spinal funiculi that are sectioned and time following injury. Neuroscience 116:1149-1160.
White, J. C., and Sweet, W. H. (1969) Pain and the neurosurgeon: a forty-year experience. (C. C. Thomas: Springfield, Ill.).
Willis, W. D., and Coggeshall, R. E. (1991) Sensory mechanisms of the spinal cord. (Plenum Press: New York).
Willis, W. D. (2002) Long-term potentiation in spinothalamic neurons. Brain Res. Brain Res. Rev. 40:202-214.
Yezierski, R. P. (2000) Pain following spinal cord injury: pathophysiology and central mechanisms. Prog. Brain Res. 129:429-449.
Zemlan F. P., Leonard, C. M., Kow, L. M., and Pfaff, D. W. (1978) Ascending tracts of the lateral columns of the rat spinal cord: a study using the silver impregnation and horseradish peroxidase techniques. Exp. Neurol. 62:298-334.
U.S. Pat. No. 4,188,398 “Method for Treating Epilepsy”
U.S. Pat. No. 4,894,376 “Methods of Treating Diseases Characterized by Hyperexcitability of Neurons”
U.S. Pat. No. 4,981,867 “Treatment of Tremor Using Compounds that Depress Calcium Currents in Thalamic and Other Central Nervous System Neurons”
U.S. Pat. No. 6,407,088 “Method of Analgesia”
U.S. Pat. No. 6,495,601 “Methods and Compositions for Treating Conditions of the Central and Peripheral Nervous Systems Using Non-Synaptic Mechanisms”

1. A method for treating central pain syndrome, comprising administering to a mammal in need thereof an effective amount of a thalamic anticonvulsant compound to treat central pain syndrome.

2. The method of claim 1, wherein said thalamic anticonvulsant compound has the formula 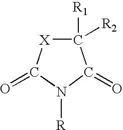
wherein: R is H or lower alkyl; R₁ and R₂ are independently selected from the group consisting of H, lower alkyl, aryl, and aryl lower alkyl; and X is —O— or —CH₂—, with the proviso that at least one of R, R₁, and R₂ is an indicated substitutent other than hydrogen.

3. The method of claim 2, wherein said thalamic anticonvulsant compound is a succinimide.

4. The method of claim 3, wherein R is H or methyl, R₁ is methyl and R₂ is methyl, ethyl or phenyl.

5. The method of claim 3, wherein the succinimide thalamic anticonvulsant compound is ethosuximide.

6. The method of claim 5, wherein said effective amount of ethosuximide is in a range from about 1 mg to about 25 mg per kilogram of said mammal.

7. The method of claim 3, wherein the succinimide thalamic anticonvulsant compound is methsuximide.

8. The method of claim 2, wherein said thalamic anticonvulsant compound is an oxazolidinedione.

9. The method of claim 8, wherein R is H or methyl, R₁ is methyl, and R₂ is methyl or ethyl.

10. The method of claim 8, wherein the oxazolidinedione thalamic anticonvulsant compound is N,5,5-trimethyloxazolidinedione (trimethadione).

11. The method of claim 8, wherein the oxazolidinedione thalamic anticonvulsant compound is N,5-dimethyl-5-ethyloxazolidinedione (paramethadione).

12. The method of claim 1, wherein said thalamic anticonvulsant compound is valproic acid.

13. The method of claim 1, wherein said mammal is a human.

14. A method for inducing centrally generated pain responses in a non-human mammal model, comprising bilaterally transecting the spinothalamic tract of a non-human mammal at the level of the thoracic/lumbar border, which severs the ascending pathways conveying somatosensory, thermal and nociceptive information to the cortex via the thalamus, to induce centrally generated pain responses in said non-human mammal.

15. The method of claim 14, wherein said step of bilaterally transecting the spinothalamic tract comprises:
performing a T8 laminectomy on an anesthesized non-human mammal;
opening the dura to expose the spinal cord;
lifting and rotating the spinal cord;
severing the ventral lateral pain pathways in the spinal cord to generate a lesion;
packing the lesion with sterile packing material; and
closing and suturing the overlying muscle and skin.

16. The method of claim 14, wherein said non-human mammal is a rat.

17. A method of screening and identifying a compound that inhibits T-type calcium channels, comprising:
detecting thalamic cell excitability in thalamic relay cells obtained from a lesion in the ventrobasal complex of the thalamus in the absence of a candidate compound;
contacting the thalamic relay cells with a candidate compound;
detecting thalamic cell excitability in the presence of the candidate compound;
identifying the candidate compound as a compound that inhibits T-type calcium channels if a reduction of thalamic cell excitability is detected in the presence of the candidate compound relative to in its absence.

18. The method of claim 17, wherein the thalamic relay cells are obtained from a rat.

19-29. (canceled)
